(19) United States
(12) Patent Application Publication
(10) Pub. No.: US 2024/0305471 A1
(43) Pub. Date: Sep. 12, 2024

(54) Universal Medical Health Records

(71) Applicant: Scott Weidley, Las Vegas, NV (US)

(72) Inventor: Scott Weidley, Las Vegas, NV (US)

(21) Appl. No.: 18/599,078

(22) Filed: Mar. 7, 2024

Related U.S. Application Data
(60) Provisional application No. 63/450,636, filed on Mar. 7, 2023.

(51) Int. Cl. H04L 9/32; G06F 21/62; G16H 10/60; H04L 9/00

(52) U.S. Cl. CPC H04L 9/3247; G06F 21/6245; G16H 10/60; H04L 9/50

(57) ABSTRACT

The system provides a method for accessing a medical record of a patient via a patient portal, wherein the medical record of the patient stored on a blockchain is provided, including receiving a primary encrypted private key associated with access of confidential data and HIPAA-compliant handling of Protected Health Information (PHI) and secondary private keys associated with an immutable audit trail, wherein the patient has the ability to manage a digital profile which can be utilized for enrollment and participation related to health insurance providers, healthcare providers, and clinical trials, as well as a general ledger for patient accessible medical records on the blockchain with interoperability among insurance companies, hospitals, doctors, doctor's offices, clinics, and clinical trials sites.

### CROSS-REFERENCE TO RELATED APPLICATIONS

The present application claims priority to U.S. Provisional Patent Application No. 63/450,636, filed Mar. 7, 2023, which is incorporated herein by reference in its entirety.

### BACKGROUND OF THE INVENTION

### Field of the Invention

The system is related to a method and apparatus for providing universal medical records. Universal medical records refer to the idea of having a single, comprehensive record, aka a single source of truth (SSOT) for a person's health information that can be accessed by any authorized healthcare provider.

### Description of the Background

The lack of uniform medical records that can be easily shared by medical professionals has been a long-time problem. For example, patient records in clinical trials are controlled by the sponsor, and not by the patient. This makes it difficult to integrate medical records from a trial with existing medical records. In the current art, these two types of medical records are not typically combined in the patient folder. In many cases, a patient's physician may have no knowledge that the patient has participated in a trial. This lack of information could be a problem in preparing a treatment plan for a patient.

Early attempts at solving these problems began with digitizing health records as Electronic Health Records (EHRs). EHRs are digital versions of a patient's paper-based medical records. EHRs allow healthcare providers to securely access and share a patient's health information. EHRs provide improved accuracy and completeness of patient information, the potential for improved patient care through access to up-to-date information, and increased efficiency in sharing information between healthcare providers. However, this improvement is limited to patient information shared between healthcare provides which is shared only in the same native healthcare system.

However, prior art EHRs have high implementation and maintenance costs. Moreover, existing EHRs have been associated with privacy and security concerns, including the risk of hacking and data breaches. The ability of EHRs to communicate outside of the native healthcare system has also been hindered by a resistance to change and adoption by healthcare providers and patients.

The prior art has also utilized National Health Information Exchanges (NHIE), which are centralized systems that allow healthcare providers to securely exchange patient information across a national level. NHIE systems allow relatively improved accuracy and completeness of patient information, increased efficiency in sharing information between healthcare providers, and improved patient care through access to up-to-date information. However, NHIE systems also have the following drawbacks: high implementation and maintenance costs, privacy and security concerns, with the risk of hacking and data breaches, and complexity associated with the coordination and standardization of information exchange across a large, diverse healthcare system.

The prior art has also utilized Personal Health Records (PHRs), which are patient-centered records that allow individuals to manage their own health information. PHRs often allow patients to access test results, medications, and other health information. However, these systems also have drawbacks, such as potential for errors and inaccuracies in self-reported information, privacy and security concerns, with the risk of hacking and data breaches, and limited use and acceptance by healthcare providers.

Lastly, the prior art has also used Interoperable Health Information Exchange (HIE) systems. These are systems that allow healthcare providers to securely share patient information across different EHR systems and other health IT systems. These systems also have drawbacks, including high implementation and maintenance costs, privacy and security concerns, with the risk of hacking and data breaches, and interoperability challenges and technical difficulties in integrating with different EHR systems.

Therefore, a system which avoids some or all of the foregoing drawbacks would be an improvement.

### SUMMARY OF THE INVENTION

Accordingly, an object of the present invention is to provide a method for accessing a medical record of a patient via a patient portal, wherein the medical record of the patient stored on a blockchain is provided.

The invention thus comprises a software enabled system for storing medical records, including Protected Health Information (PII), using multiple keys controlled by the patient/owner of the medical records. The software-enabled system performs steps including receiving a primary encrypted private key associated with access of confidential data and HIPAA-compliant handling of Protected Health Information (PHI) and secondary private keys associated with an immutable audit trail, wherein the patient has the ability to manage a digital profile which can be utilized for enrollment and participation related to health insurance providers, healthcare providers, and clinical trials, as well as a general ledger for patient accessible medical records on the blockchain with interoperability among insurance companies, hospitals, doctors, doctor's offices, clinics, and clinical trials sites.

The foregoing objects, features and attendant benefits of this invention will, in part, be pointed out with particularity and will become more readily appreciated as the same become better understood by reference to the following detailed description of a preferred embodiment and certain modifications thereof when taken in conjunction with the accompanying example formulations.

### BRIEF DESCRIPTION OF THE DRAWINGS

FIG. 1 is a block diagram of an embodiment of the system.

FIG. 2 is a block diagram of a blockchain transaction of the system in an embodiment.

FIG. 3 is a flow diagram of a method of accessing a medical record via a portal in an embodiment of the system.

### DETAILED DESCRIPTION OF THE PREFERRED EMBODIMENTS

The present invention is a software-enabled system for securely accessing a patient's medical record through a patient portal. The patient's medical record is stored on a decentralized blockchain, ensuring transparency and interoperability across healthcare providers, insurance companies, and clinical trials sites. Access to confidential data is governed by a primary encrypted private key, maintaining HIPAA compliance and safeguarding Protected Health Information (PHI). Additionally, the patient retains control over a tokenized digital profile, which enables easy enrollment and participation in various healthcare services and clinical trials. Secondary private keys are assigned to providers, ensuring secure management of enrollment and participation in these services, which is also tokenized. Finally, an immutable audit trail is available on the blockchain, enhancing transparency and accountability within the system, while protecting PHI via anonymity.

The system implements patient medical records of a patient on a blockchain. The system also acts as a portal for enrolment in healthcare insurance, participation with healthcare providers, and recruitment for clinical trials via the digitization, decentralization, and democratization of patient medical records, creating full interoperability at insurance companies, hospitals, physician's offices, clinics, and clinical trial sites.

Blockchain systems are known in the art, however they have various drawbacks, including technical difficulties in implementation and adoption, limited scalability and performance in handling large amounts of health data, and regulatory and legal challenges in the use of decentralized systems.

By contrast, the present invention enables the secure, HIPPA compliant storage of healthcare data including PII, via the use of a threefold encryption system: a primary encrypted private key and a tokenized digital profile, both held by the patient, and secondary private keys assigned to providers.

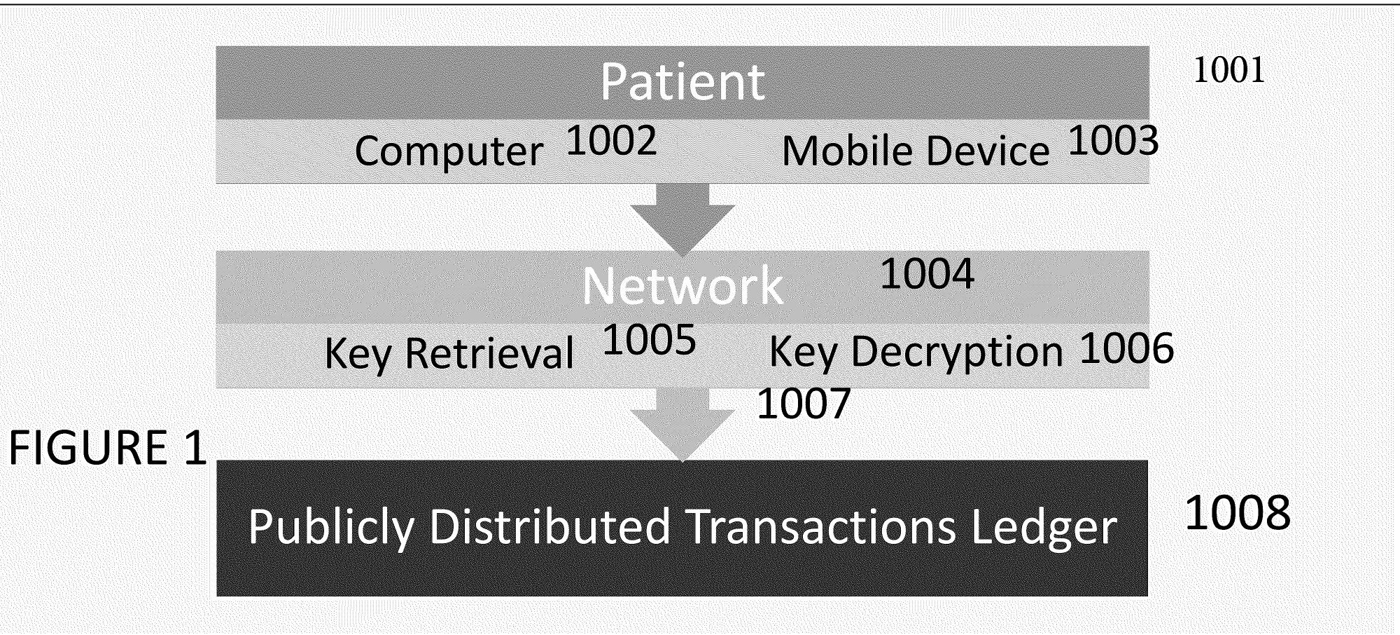

FIG. 1 illustrates a functional block diagram of the system. A Patient 1001 can access the system via a computing system 1002 or mobile device 1003. The Patient 1001 communicates through Network 1004 for Key Retrieval 1005 and Key Decryption 1006. Data 1007 is communicated to Publicly Distributed Transactions Ledger 1008, which stores the Patient Record on a blockchain or other immutable storage system.

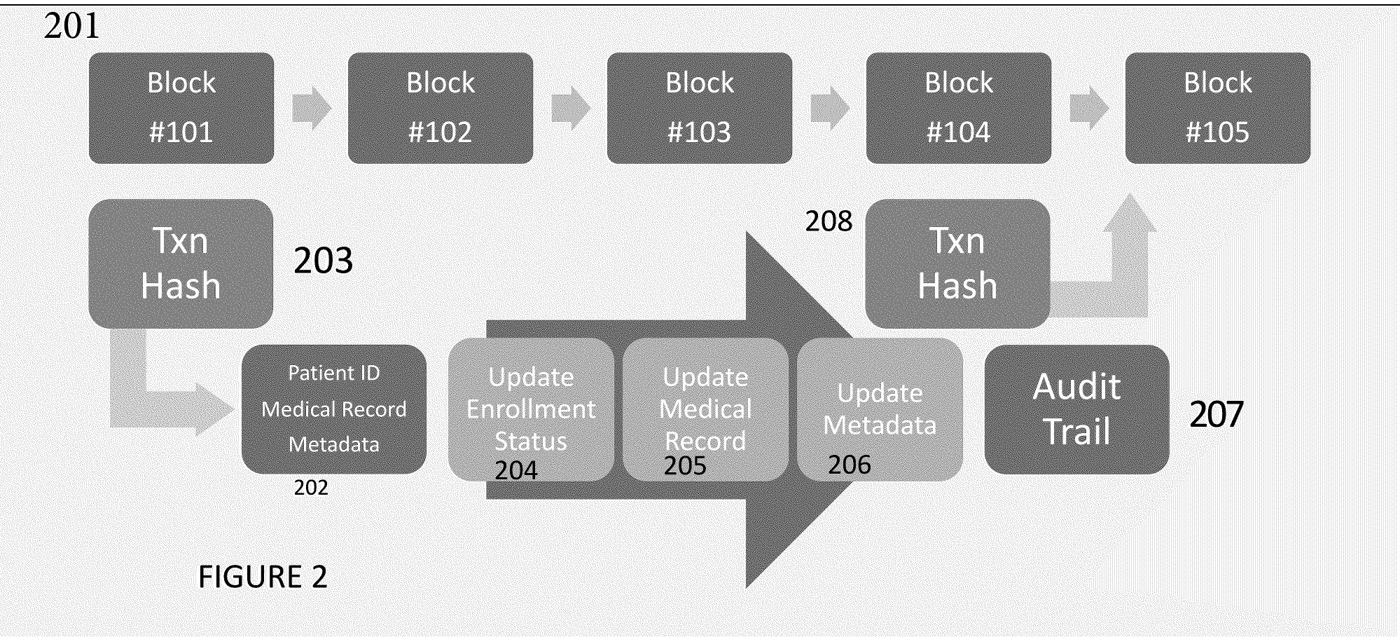

FIG. 2 is a block diagram of a blockchain transaction in an embodiment. Blockchain 201 is comprised of blocks 101, 102, 103, 104, and 105. When Patient ID Medical Record Metadata 202 is accessed a Transaction Hash 203 (Txn) validates the access. The authorized user can then update enrolment status 204, update medical records 205, and update metadata 206, among other changes that can be made to the record.

An audit trail 207 is automatically created and the updates are written to the blockchain (e.g. block #105) and another Txn 208 validates the transaction.

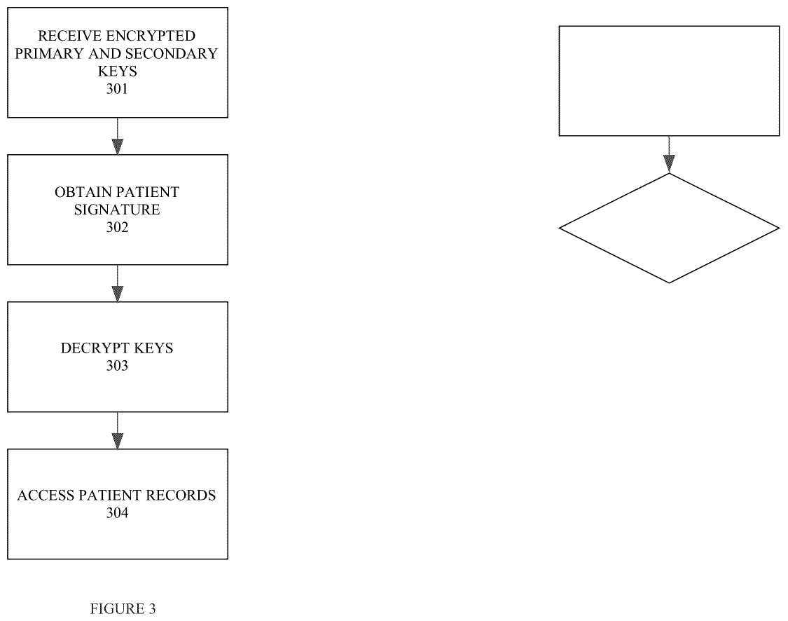

FIG. 3 is a flow diagram illustrating a method of accessing a medical record in an embodiment of the system. At step 301 the system receives a primary encrypted private key for the Patient and a secondary encrypted private key for the healthcare providers. At step 302 the system requests and obtains the digital signature of the Patient for decryption the keys. At step 303 the system decrypts the primary key and the secondary key using the signature of the Patient. At step 304 the Patient is able to now access the medical records. The patient access is relative to confidential and HIPAA-compliant protocols for Protected Health Information (PHI). The system provides an immutable audit trail using the primary and secondary keys.

In one embodiment, the system uses an Ethereum blockchain technology, such as a wallet browser plugin or add-on. The Patient and other users can store ERC-20 compliant tokens enabling the users to enact with any Ethereum address. It will be understood that any suitable blockchain technology or other immutable storage system may be used without departing from the scope and spirit of the present system.

The system provides an interface to the blockchain to allow account management and connectivity without running the Ethereum node software locally. This is done by using a web-based wallet. This web-based wallet provides a way to interact with the Ethereum blockchain without needing to run a node. It provides an interface to the blockchain, allowing users to send and receive Ether and interact with smart contracts. Decentralized exchanges also provide a way to interact with the Ethereum blockchain without running a node, as these exchanges also allow users to interact with smart contracts. By using a decentralized exchange, users can manage their accounts on the blockchain without running an Ethereum node locally. While this system will utilize a web-based wallet, it will be understood that a decentralized exchange may also be used without departing from the scope and spirit of the present system.

The system portal provides access to decentralized applications that combine smart contracts (e.g. utilizing Solidity) and a custom user interface.

The description and illustrations are by way of example only. While the description above makes reference to various embodiments, it should be understood that many changes and modifications can be made without departing from the scope of the disclosure. Many more embodiments and implementations are possible within the scope of this invention and will be apparent to those of ordinary skill in the art. The invention is not limited to the specific details, representative embodiments, and illustrated examples in this description.

I claim:

1. A method for accessing a medical record of a patient via a patient portal, wherein the medical record of the patient is stored on a blockchain, comprising:
receiving a primary encrypted private key for the patient and secondary encrypted private keys for the healthcare providers, wherein the encrypted private keys are decrypted by signature of the patient;
obtaining the signature of the patient for decryption of the private keys;
decrypting the encrypted private keys using the signature of the patient to determine a private key associated with the patient; and
accessing the medical records of the patient, using a combination of the private key associated with the patient, relative to confidential data and HIPAA-compliant handling of Protected Health Information (PHI), and secondary private keys, relative to an immutable audit trail.
